(19) United States
(12) Patent Application Publication
(10) Pub. No.: US 2026/0232549 A1
(43) Pub. Date: Aug. 13, 2026

(54) Oral Care Compositions

(71) Applicant: Colgate-Palmolive Company, New York, NY (US)

(72) Inventors: Rong DONG, Highland Park, NJ (US); Suman CHOPRA, Monroe, NJ (US); Gregory SZEWCZYK, Flemington, NJ (US)

(73) Assignee: Colgate-Palmolive Company, New York, NY (US)

(21) Appl. No.: 19/155,982

(22) PCT Filed: Feb. 13, 2024
(86) PCT No.: PCT/US2024/015592

§ 371 Date: Aug. 13, 2025

Related U.S. Application Data
(60) Provisional application No. 63/445,274, filed on Feb. 13, 2023.

(51) Int. Cl. A61K 8/22; A61K 8/24; A61K 8/25; A61K 8/46; A61K 8/81; A61K 8/86; A61Q 11/00

(52) U.S. Cl. CPC A61K 8/22; A61K 8/24; A61K 8/25; A61K 8/463; A61K 8/8176; A61K 8/86; A61Q 11/00; A61K 2800/28; A61K 2800/30; A61K 2800/48

(57) ABSTRACT

Described herein are aqueous abrasive oral care compositions comprising (a) 1-10 wt. % hydrogen peroxide, (b) 5-20 wt. % of calcium pyrophosphate abrasive, (c) 35-75 wt. % propylene glycol, (d) 5-20 wt. % polyvinylpyrrolidone, (e) 1-20 wt. % of a random polyethylene glycol/polypropylene glycol random copolymer. Methods of making and using these compositions are also described herein.

### CROSS-REFERENCE TO RELATED PATENT APPLICATIONS

This application claims priority to U.S. Provisional Patent Application No. 63/445,274, entitled, “Oral Care Compositions,” filed on Feb. 13, 2023, the content of which is incorporated herein by reference in its entirety for all purposes.

### BACKGROUND

Many individuals desire a “bright” smile and white teeth, and consider dull and stained teeth cosmetically unattractive. Thus, there is a desire for whiter teeth and one means to achieve whiter teeth is the use of tooth whitening products.

Teeth can become discolored by foods, drinks and tobacco use. Dental stains can be classified as either extrinsic, which occur on the outer surface of teeth, or intrinsic, which occur below the surface of enamel. Most abrasive containing toothpaste remove extrinsic stains. Peroxide oxidizing agents can bleach both extrinsic and intrinsic stains and so provide fast and superior whitening efficacy compared to abrasive methods. In addition to bleaching the teeth and removing stains, peroxide agents also kill cariogenic bacteria.

There are numerous peroxide sources available commercially, including solid inorganic peroxides, polymer-bound peroxides, and hydrogen peroxide. Hydrogen peroxide is the simplest peroxide compound, and among the cheapest peroxide agents available. It is widely used as a bleaching agent (e.g., paper bleaching), as an industrial antiseptic (e.g., in sewage treatment), and as a consumer antiseptic. It is commonly available as an aqueous solution ranging in concentration from 5% to 70%.

However, hydrogen peroxide is an unstable molecule, and it undergoes decomposition to form oxygen gas and water. Because decomposition produces gaseous oxygen, in a closed container it can cause the container to leak, split, or burst from the pressure. While it normally decomposes slowly, rapid and dangerous decomposition can occur in the presence of trace amounts of many metals or organic compounds, and can be promoted by heat. Decomposition is also suppressed by acidic conditions and accelerated by basic conditions, and it is thus highly pH-dependent. While aqueous hydrogen peroxide has good stability at pH 3.5-4.5, above pH 5 the rate of aqueous hydrogen peroxide decomposition rapidly rises.

Common abrasives used in dentifrice compositions are also particularly problematic for hydrogen peroxide. Most silica and alumina abrasives in particular have inextricable trace quantities of metals present, which are sufficient to catalyze rapid decomposition of hydrogen peroxide to oxygen and water. Toothpaste tubes formulated with abrasive silica or alumina have been known to explode as soon as one day after filling. This is particularly true for the so-called “high-cleaning silica” abrasives.

Hydrogen peroxide decomposition is also exothermic, and since heat promotes decomposition, this can result in an uncontrolled autocatalytic decomposition. For aqueous hydrogen peroxide, the higher the concentration of hydrogen peroxide, the more exothermic the decomposition will become. For high concentrations, such as above 70% hydrogen peroxide, decomposition can generate enough heat to reach the boiling point of the solution (110-140° C.). Thus, highly concentrated aqueous hydrogen peroxide is potentially very dangerous. The stability of aqueous hydrogen peroxide is increased by keeping the pH low (<5) and by including various stabilizing agents in the solution which suppress decomposition (stannate salts and mineral acids are common).

Commonly available concentrations of aqueous hydrogen peroxide include 3-6% (used as consumer or medical antiseptic), 30-35% (most common for laboratory use and as an industrial disinfectant), 50-70% (used industrially), and 80-90% (used as a rocket fuel).

It has long been a great challenge to formulate stable dentifrice compositions using hydrogen peroxide. As a result, hydrogen peroxide has been conventionally avoided in the oral care industry due to the numerous manufacturing and stability difficulties resulting from the reactivity of hydrogen peroxide. In small, soft-walled containers, such as toothpaste tubes, even a small degree of gas accumulation from hydrogen peroxide decomposition can be problematic in storage, causing dentifrice containers to bloat, burst, or leak. Additionally, because the hydrogen peroxide content of dentifrice formulations is tightly controlled for optimum whitening ability, the degradation of even a portion of the hydrogen peroxide in a dentifrice composition may cause the remaining formulation to have insufficient oxidizing capacity to clean and whiten teeth effectively.

One method used to address the stability issues of hydrogen peroxide is to simply formulate a dentifrice with very high levels of hydrogen peroxide, with the expectation that it will decompose over time. However, this results in the exact amount of peroxide delivered on application of the dentifrice to be highly variable and largely dependent on how long and under what conditions the dentifrice has been stored.

Another method is to use strictly anhydrous dentifrice compositions, because in the complete absence of water, hydrogen peroxide stability is improved. However, in the absence of water, it can become quite difficult to formulate a dentifrice that is both effective and has pleasing physical characteristics. Many of the functionally important ingredients of a dentifrice, such as fluoride salts and phosphate salts, are very water soluble, but poorly soluble or insoluble in a non-aqueous environment. In addition, many gelling agents commonly used in dentifrice formulations are also susceptible to oxidation by aqueous hydrogen peroxide, creating a difficult formulation scenario for HP dentifrices—with water the gelling agents function well, but may oxidize, and without water they are safe from oxidation but may function poorly.

Another method is to rely on non-abrasive dentifrice formulas, but these suffer from the drawback that the abrasives are very desirable in a whitening composition due to their ability to debride and physically scrub the external surface of the teeth, greatly contributing to the removal of superficial stains and discolorations, as well as microabrading the enamel surface increasing its luster (shine). Thus, non-abrasive dentifrice formulas will lack all of these features, which are particularly complementary to the whitening effect provided by peroxide.

Other common dentifrice ingredients have also been reported to be unstable in the presence of hydrogen peroxide, or to destabilize hydrogen peroxide, although there have been some conflicting accounts. U.S. Pat. No. 4,839,156 reports that anionic surfactants and many common thickening agents (including alginates and celluloses) are unstable in the presence of hydrogen peroxide, and that the use of PVP results in stringy gels, rather than the desirable homogenous rigid gels.

Another approach is shown by U.S. Pat. No. 5,851,514, which discloses an abrasive aqueous whitening dentifrice which requires a particular selection of polymers and humectants (polyethylene glycol, poloxamers, and glycerin), in a high-water vehicle (15-40% water), stabilized by the addition of a metal scavenging agents (chelating agents, such as metal stannate salts or ethylenediaminetetraacetic acid (EDTA) or its salts). Indeed, the metal scavenging agent is critical to the stability of these high-water compositions, and it is pre-mixed with the aqueous hydrogen peroxide (35%) prior to combination with other components.

There have also been some reports suggesting that polyethylene glycol can be oxidized by hydrogen peroxide to generate dioxane, and therefore, there is reason to avoid formulating oral care compositions which combine aqueous hydrogen peroxide (non-complexed) with a vehicle comprising polyethylene glycol.

Another strategy that has been attempted is the use of dual-component compositions, in which the hydrogen peroxide agent (e.g., aqueous hydrogen peroxide solution or gel) is sequestered from the ingredients that might destabilize it (e.g., abrasives) until the point of mixing and application to the teeth. However, this results in much more expensive dispensing devices and complicates manufacturing processes as, in essence, two separate compositions must be manufactured and packaged.

In alternative methods, the pH of an aqueous hydrogen peroxide composition is decreased to an acidic range in order to increase H₂O₂ stability, but this has an adverse effect on the taste of the composition (acidic materials having a strong sour taste).

Over the last 10-15 years, the leading peroxide-based tooth whitening dentifrice composition has comprised a pre-formed complex between cross-linked polyvinylpyrrolidone (cPVP) and hydrogen peroxide (often called “cPVP-H₂O₂ complex” or simply “PVP-HP complex”). The complex contains about 18-20 weight % of hydrogen peroxide. As described in U.S. Pat. Nos. 9,517,194, and 5,108,742, cPVP-HP complex is made by suspending cPVP in a suitable anhydrous organic solvent, preferably anhydrous ethyl acetate, and then adding an anhydrous solution of hydrogen peroxide, also preferably in anhydrous ethyl acetate, and upon mixing a solid precipitate of the complex forms.

The cPVP-HP complex, sold under brand names such as Peroxydone, is available as a powdered solid with good solubility in organic solvents and hydrophobic polymers, but is generally not water soluble. The complex is formulated into an anhydrous composition based on silicone polymers and other hydrophobic agents. PVP is a fairly hydrophobic polymer, but it includes the hydrophilic amide functional group in the lactam ring of the repeating unit, and hydrogen peroxide molecules can complex to the lactam ring. However, the polymer backbone is hydrophobic, and so formation of the complex helps stabilize the hydrogen peroxide from degradation. One of the drawbacks to using PVP-HP complex is that its commercial availability is limited, and its cost is very high. In addition, in order to formulate a composition having, for example, 4 weight % hydrogen peroxide, the composition would have to comprise about 20 weight % PVP-HP complex, and thus, about 16 weight % PVP from the complex. Because PVP has thickening properties, it can become difficult to formulate a dentifrice with this high amount of PVP in addition to other necessary components which also have thickening properties.

Accordingly, there is a need for improved aqueous abrasive whitening oral care compositions having stable hydrogen peroxide formulations, acceptable taste, and shelf-stability. Accordingly, embodiments of the present invention are designed to provide these, and other, benefits.

### BRIEF SUMMARY

The present disclosure relates to oral care composition and methods of producing and using the same.

The present disclosure provides an aqueous abrasive oral care composition comprising (a) 1-10 wt. % hydrogen peroxide, (b) 5-20 wt. % of calcium pyrophosphate abrasive, (c) 35-75 wt. % propylene glycol, (d) 5-20 wt. % polyvinylpyrrolidone, (e) 1-20 wt. % of a polyethylene glycol/polypropylene glycol random copolymer;
provided that:
(1) the hydrogen peroxide is incorporated into the composition as a 40-80 wt. % aqueous solution (e.g., 50 wt. % aqueous hydrogen peroxide); or
(2) the hydrogen peroxide is incorporated into the composition as a 30-80 wt. % aqueous solution (e.g., 50 wt. % aqueous hydrogen peroxide) and the composition is not formulated with metal ion chelating agents, such as metal stannate salts, colloidal stannate, or EDTA or its salts.

In further aspects, the present disclosure provides a method of making the composition, and methods of using the composition for whitening and/or cleaning the teeth.

According to another aspect of the invention, provided is a kit. The kit typically includes a container and an oral care composition disposed in the container.

### BRIEF DESCRIPTION OF THE DRAWINGS

FIGURE is a FTIR spectroscopy graph of a region from 1550 to 1750 cm⁻¹ for a non-limiting example oral care composition, a comparative oral care composition, and a pure PVP polymer according to aspects of the invention.

### DETAILED DESCRIPTION

The inventors discovered that oral care compositions disclosed herein can advantageously maintain the stability of the peroxide, reduce the amount of thickening system, and, preferably, improve the flavor profile and texture of such oral care composition. For instance, certain oral care compositions disclosed herein surprisingly achieve a high level of peroxide stability with a reduced amount of thickening agents while maintaining the levels of abrasives, fluoride, and other components that are traditionally believed to degrade the peroxide level. The inventors were also unexpectedly able to reduce the total amount of thickening agents while maintaining a desirable viscosity in certain embodiments.

Additionally, the inventors discovered that certain oral care compositions prepared with a hydrogen peroxide aqueous solution, certain thickening systems comprising particular amounts of thickening agents, certain abrasives, and at particular pHs surprisingly achieve enhanced flavor profiles (while using the same flavoring agents at the same concentration) and an improved texture. The oral care compositions may surprisingly achieve one or more of the benefits described herein while having a reduced amount or being free of fatty amphiphiles.

In a first aspect, the present disclosure provides an aqueous abrasive oral care composition (Composition 1) comprising (a) 1-10 wt. % hydrogen peroxide, (b) 5-20 wt. % of calcium pyrophosphate abrasive, (c) 35-75 wt. % propylene glycol, (d) 5-20 wt. % polyvinylpyrrolidone, (e) 1-20 wt. % of a polyethylene glycol/polypropylene glycol random copolymer;
provided that:
   (1) the hydrogen peroxide is incorporated into the composition as a 40-80 wt. % aqueous solution (e.g., 50 wt. % aqueous hydrogen peroxide); or
   (2) the hydrogen peroxide is incorporated into the oral care composition as a 30-80 wt. % aqueous hydrogen peroxide solution (e.g., 50 wt. % aqueous hydrogen peroxide) and the oral care composition is not formulated with metal ion chelating agents, such as metal stannate salts, colloidal stannate, or EDTA or its salts.

In further embodiments of the first aspect, the present disclosure provides:
1.1 Composition 1, wherein the hydrogen peroxide is incorporated into the oral care composition as an about 40-80 wt. % aqueous hydrogen peroxide solution, e.g., an about 40-70 wt. % aqueous hydrogen peroxide solution, an about 40-60 wt. % aqueous hydrogen peroxide solution, or an about 45-55 wt. % aqueous hydrogen peroxide solution. In other words, the aqueous hydrogen peroxide solution may comprise about 40-80 wt. % hydrogen peroxide, about 40-60 wt. % hydrogen peroxide, about 45-55 wt. % hydrogen peroxide, wherein the weight percentage is based on the total weight of the aqueous hydrogen peroxide solution.
1.2 Composition 1, wherein the hydrogen peroxide is incorporated into the oral care composition as an about 30-80 wt. % aqueous hydrogen peroxide solution, e.g., an about 30-70 wt. % aqueous hydrogen peroxide solution, an about 30-60 wt. % aqueous hydrogen peroxide solution, an about 40-60 wt. % aqueous hydrogen peroxide solution, or an about 45-55 wt. % aqueous hydrogen peroxide solution. In other words, the aqueous hydrogen peroxide solution may comprise about 30-80 wt. % hydrogen peroxide, about 30-70 wt. % hydrogen peroxide, about 30-36 wt. % hydrogen peroxide, wherein the weight percentage is based on the total weight of the aqueous hydrogen peroxide solution.
1.3 Composition 1.1 or 1.2, wherein the hydrogen peroxide is incorporated into the composition as an about 50% aqueous solution. In other words, the aqueous hydrogen peroxide solution may comprise about 50 wt. % hydrogen peroxide, wherein the weight percentage is based on the total weight of the aqueous hydrogen peroxide solution.
1.4 Composition 1, or any of 1.1-1.3, wherein the oral care composition comprises 1-6 wt. % hydrogen peroxide, e.g., 1-5 wt. %, 1-4 wt. %, 1-3 wt. %, 2-6 wt. %, 2-5 wt. %, 2-4 wt. %, 2-3 wt. %, 3-6 wt. %, 3-5 wt. %, 3-4 wt. %, about 1 wt. %, about 2 wt. %, about 3 wt. %, about 4 wt. %, about 5 wt. %, or about 6 wt. %, hydrogen peroxide, relative to the total weight of the oral care composition (e.g., at the time of manufacture of the oral care composition).
1.5 Composition 1, or any of 1.1-1.4, wherein the only added water in the oral care composition is the water from the aqueous hydrogen peroxide.
1.6 Composition 1, or any of 1.1-1.5, wherein the oral care composition comprises 1-10% water, e.g., 1-8 wt. %, 1-6 wt. %, 1-5 wt. %, 1-4 wt. %, 1-3 wt. %, 2-6 wt. %, 2-5 wt. %, 2-4 wt. %, 2-3 wt. %, 3-6 wt. %, 3-5 wt. %, 3-4 wt. %, about 1 wt. %, about 2 wt. %, about 3 wt. %, about 4 wt. %, about 5 wt. %, or about 6 wt. % of water, relative to the total weight of the oral care composition.
1.7 Composition 1, or any of 1.1-1.6, wherein the oral care composition comprises about an equal amount of water and hydrogen peroxide by weight (e.g., at the time of manufacture of the composition). The oral care composition may comprise an aqueous hydrogen peroxide solution having a weight ratio of the hydrogen peroxide to the water from about 1:5 to about 5:1. For instance, the weight ratio of the hydrogen peroxide to the water may be about 1:2 to about 2:1. In at least one embodiment, the weight ratio of the hydrogen peroxide to the water is about 1:1.
1.8 Composition 1, or any of 1.1-1.7, wherein the oral care composition is not formulated using a PVP-hydrogen peroxide complex (e.g., a cPVP-hydrogen peroxide complex), i.e., PVP-hydrogen peroxide complex is not added to the oral care composition during manufacture.
1.9 Composition 1, or any of 1.1-1.8, wherein the oral care composition does not have any other peroxide whitening agents, such as peroxide salts (e.g., sodium peroxide), hydroperoxides (e.g., tert-butyl hydroperoxide), organic peroxides (e.g., benzoyl peroxide), or peroxy acids (e.g., peracetic acid).
1.10 Composition 1, or any of 1.1-1.9, wherein the oral care composition further comprises a peroxysulfate salt (e.g., potassium peroxymonosulfate) as the only other oxidizing agent.
1.11 Composition 1, or any of 1.1-1.9, wherein the oral care composition does not comprise any peroxysulfate salts (e.g., peroxymonosulfates or peroxydisulfates).
1.12 Composition 1, or any of 1.1-1.9, wherein the oral care composition does not comprise any of the following: urea peroxide, carbamide peroxide, peroxide salts, peroxy acids, organic peroxides, perborate salts, persilicate salts, percarbonate salts, chlorinated oxidizing agents, peroxymonosulfuric acid or peroxymonosulfate salts, or peroxydisulfuric acid or peroxydisulfate salts.
1.13 Composition 1, or any of 1-1.1.9, wherein the aqueous hydrogen peroxide solution is the only oxidizing agent in the composition.
1.14 Composition 1, or any of 1.1-1.13, wherein the oral care composition further comprises a non-oxidative whitening agent (e.g., a dye or pigment, such as blue dye or pigment, or a white pigment, such as titanium dioxide).
1.15 Composition 1, or any of 1.1-1.14, wherein the oral care composition comprises 6 to 18 wt. % of the calcium pyrophosphate abrasive, e.g., 8 to 18 wt. %, or 10 to 18 wt. %, or 12 to 18 wt. %, or 13 to 17 wt. %, or about 15 wt. %, relative to the total weight of the oral care composition.
1.16 Composition 1, or any of 1.1-1.15, wherein the oral care composition has not more than 5 wt. % of any abrasive silica, e.g., not more than 4 wt. %, or not more than 3 wt. %, or not more than 2 wt. %, or not more than 1 wt. %, relative to the weight of the oral care composition. In some embodiments, the oral care composition comprises an abrasive silica (e.g., high-cleaning silica) in an amount from about 0.1 to about 4 wt. %, about 0.1 to about 3 wt. %, about 0.1 to about 2 wt. %, about 0.1 to about 1 wt. %, about 0.1 to about 0.5 wt. %; from wt. %, relative to the total weight of the oral care composition.
1.17 Composition 1, or any of 1.1-1.15, wherein the oral care composition does not comprise high-cleaning silica.
1.18 Composition 1, or any of 1.1-1.15, wherein the oral care composition does not comprise precipitated silica or hydrated silica.
1.19 Composition 1, or any of 1.1-1.18, wherein the oral care composition does not comprise alumina.
1.20 Composition 1, or any of 1.1-1.19, wherein the oral care composition does not comprise sodium metaphosphate, calcium carbonate, calcium orthophosphate, tricalcium phosphate, dicalcium orthophosphate, sodium carbonate, sodium bicarbonate, arginine carbonate, or arginine bicarbonate.
1.21 Composition 1, or any of 1.1-1.20, wherein the calcium pyrophosphate is the only abrasive in the oral care composition.
1.22 Composition 1, or any of 1.1-1.21, wherein the oral care composition comprises 40-70 wt. %, 45-70 wt. %, 50-70 wt. %, 40-65 wt. %, 45-65 wt. %, 50-65 wt. %, 40-60 wt. %, 45-60 wt. %, 50-60 wt. %, 40-55 wt. %, 45-55 wt. %, 50-55 wt. %, about 50 wt. % propylene glycol by weight of the oral care composition.
1.23 Composition 1, or any of 1.1-1.22, wherein the oral care composition does not comprise glycerol.
1.24 Composition 1, or any of 1.1-1.23, wherein the oral care composition does not comprise sorbitol, xylitol, ethylene glycol, butylene glycol, pentylene glycol, hexylene glycol, 1,3-propanediol, diethylene glycol, dipropylene glycol, caprylyl glycol, or glycerol.
1.25 Composition 1, or any of 1.1-1.24, wherein the propylene glycol is the only polyol humectant in the oral care composition (e.g., the only polyhydroxylated non-polymeric small molecule).
1.26 Composition 1, or any of 1.1-1.25, wherein the oral care composition comprises 6-18 wt. % of polyvinylpyrrolidone, e.g., 6 to 16 wt. %, or 6 to 14 wt. %, or 7 to 16 wt. %, or 8 to 16 wt. %, or 8 to 15 wt. %, or 8 to 14 wt. %, or 8 to 12 wt. %, or 9 to 12 wt. %, or 9 to 11 wt. %, or 9 to 10 wt. %, or about 9 wt. %, or about 10 wt. %, or about 9.5 wt. % of polyvinylpyrrolidone. For example, The polyvinylpyrrolidone may be present in the oral care compositions in an amount from about 6 to about 18 wt. %, about 6 to about 16 wt. %, about 6 to about 14 wt. %, about 6 to about 12 wt. %, about 6 to about 11 wt. %, about 6 to about 10 wt. %, about 6 to about 9 wt. %, about 6 to about 8 wt. %; from about 7 to about 18 wt. %, about 7 to about 16 wt. %, about 7 to about 14 wt. %, about 7 to about 12 wt. %, about 7 to about 11 wt. %, about 7 to about 10 wt. %, about 7 to about 9 wt. %; from about 8 to about 18 wt. %, about 8 to about 16 wt. %, about 8 to about 14 wt. %, about 8 to about 12 wt. %, about 8 to about 11 wt. %, about 8 to about 10 wt. %, about 8 to about 9 wt. %; from about 9 to about 18 wt. %, about 9 to about 16 wt. %, about 9 to about 14 wt. %, about 9 to about 12 wt. %, about 9 to about 11 wt. %, about 9 to about 10 wt. %; from about 10 to about 18 wt. %, about 10 to about 16 wt. %, about 10 to about 14 wt. %, about 10 to about 12 wt. %; from about 12 to about 18 wt. %, about 12 to about 16 wt. %, about 12 to about 14 wt. %; about 16 to about 18 wt. %, or any range or subrange thereof, based on the total weight of the oral care composition.
1.27 Composition 1, or any of 1.1-1.26, wherein the polyvinylpyrrolidone is cross-linked polyvinylpyrrolidone.
1.28 Composition 1, or any of 1.1-1.26, wherein the polyvinylpyrrolidone comprises a mixture of linear and cross-linked polyvinylpyrrolidone.
1.29 Composition 1, or any of 1.1-1.27, wherein the oral care composition does not comprise linear polyvinylpyrrolidone.
1.30 Composition 1, or any of 1.1-1.29, wherein the hydrogen peroxide and the polyvinylpyrrolidone form a polyvinylpyrrolidone-hydrogen peroxide complex in situ during manufacture of the composition.
1.31 Composition 1.30, wherein the polyvinylpyrrolidone-hydrogen peroxide complexed formed in-situ has a mass ratio of polyvinylpyrrolidone to hydrogen peroxide of about 9:1 to about 1.5:1, e.g., 5:1 to 1.5:1, or 4:1 to 1.5:1, or 3:1 to 1.5:1, or 2.5:1 to 1.5:1, or 2.25:1 to 1.5:1, or 2:1 to 1.5:1, or 1.8:1 to 1.5:1, or about 4.5:1, or about 3:1, or about 2.25:1, or about 1.8:1.
1.32 Composition 1, or any of 1.1-1.31, wherein the oral care composition comprises 5 to 20 wt. % of a polyethylene glycol/polypropylene glycol (PEG/PPG) random copolymer, e.g., 5 to 18 wt. %, or 5 to 15 wt. %, or 5 to 12 wt. %, or 5 to 10 wt. %, or 7 to 15 wt. %, or 7 to 12 wt. %, or 7 to 10 wt. %, or 7 to 9 wt. %, or 7 to 8 wt. %, or 7.5 to 10 wt. %, or about 7.5 wt. %, or about 10 wt. %, relative to the total weight of the oral care composition.
1.33 Composition 1, or any of 1.1-1.32, wherein the PEG/PPG random copolymer has an average molar ratio of ethylene glycol units (EG) to propylene glycol units (PG) of about 75-150 EG to 45-95 PG, or about 95-135 EG to 50-80 PG, or about 105-125 EG to 55-75 PG, or about 110-120 EG to 60-70 PG, or about 116 EG to 66 PG (i.e., PEG/PPG 116/66), optionally wherein the polymer has an average molecular weight of 8000 to 13,000 Da, e.g., 9000 to 10,000 Da or about 9800 Da.
1.34 Composition 1.33, wherein the PEG/PPG random copolymer is PEG/PPG 116/66 (e.g., Pluracare L1220).
1.35 Composition 1, or any of 1.1-1.34, wherein the oral care composition is not formulated with metal ion chelating agents.
1.36 Composition 1.35, wherein said metal ion chelating agents are metal stannate salts (e.g., sodium stannate, potassium stannate, lithium stannate, calcium stannate, magnesium stannate), colloidal stannate, EDTA, disodium EDTA, tetrasodium EDTA, dipotassium EDTA, tetrapotassium EDTA, or the like.
1.37 Composition 1, or any of 1.1-1.36, wherein the oral care composition comprises not more than 0.1 wt. % of metal ion chelating agents (e.g., metal stannate salts and EDTA or its salts), e.g., less than 0.05 wt. %, less than 0.01 wt. %, less than 0.005 wt. %, less than 0.001 wt. %, less than 0.0005 wt. %, or less than 0.0001%, relative to the total weight of the oral care composition.
1.38 Composition 1, or any of 1.1-1.36, wherein the composition has less than 0.01 wt. % of metal ion chelating agents (e.g., metal stannate salts and EDTA or its salts), relative to the total weight of the oral care composition.
1.39 Composition 1, or any of 1.1-1.36, wherein the oral care composition has less than 0.01 wt. % of any metal stannate salts, EDTA or its salts, relative to the total weight of the oral care composition.
1.40 Composition 1, or any of 1.1-39, wherein the oral care composition further comprises fumed silica as a thickening agent.
1.41 Composition 1.40, wherein the oral care composition comprises 1-6 wt. % of fumed silica, e.g., 1-5 wt. %, or 1.5-5.5 wt. %, or 2-5 wt. %, or 3-5 wt. %, or 4-5 wt. %, or 1-4 wt. %, or 2-4 wt. %, or 3-4 wt. %, of fumed silica, based on the total weight of the oral care composition. For instance, the oral care composition may include fumed silica in an amount from about 1 to about 5 wt. %, about 1 to about 4 wt. %, about 1 to about 3.5 wt. %; from about 2 to about 5 wt. %, about 2 to about 4 wt. %, about 2 to about 3.5 wt. %; from about 3 to about 5 wt. %, about 3 to about 4 wt. %, about 3 to about 3.5 wt. %, or any range or subrange thereof, based on the total weight of the oral care composition.
1.42 Composition 1, or any of 1.1-1.41, wherein the oral care composition further comprises one or more surfactants, e.g., anionic surfactants, cationic surfactants, amphoteric, non-ionic, and/or zwitterionic surfactants.
1.43 Composition 1.42, wherein the oral care composition comprises an anionic surfactant.
1.44 Composition 1.43, wherein the anionic surfactant is selected from: sodium lauryl sulfate, sodium laureth sulfate, sodium myreth sulfate, sodium lauroyl sarcosinate, sodium coconut monoglyceride sulfonates, sodium lauryl benzene sulfonate, sodium lauryl sulfoacetate, sodium N-methyl N-cocoyl taurate, sodium cocoyl isethionate, sodium dioctyl sulfosuccinate, and sodium cocomonoglyceride sulfate, and ammonium analogs thereof.
1.45 Composition 1.45, wherein the anionic surfactant is sodium lauryl sulfate.
1.46 Any of Compositions 1.42-1.45, wherein the oral care composition comprises 1-5 wt. % of the anionic surfactant (e.g., sodium lauryl sulfate), e.g., 1-4 wt. %, 1-3 wt. %, 2-3 wt. %, or about 2 wt. % of anionic surfactant (e.g., about 2 wt. % sodium lauryl sulfate), relative to the total weight of the oral care composition.
1.47 Any of Compositions 1.42-1.46, wherein the oral care composition further comprises a cationic surfactant, amphoteric surfactant, non-ionic surfactant, and/or zwitterionic surfactant.
1.48 Any of Compositions 1.42-1.47, wherein said zwitterionic surfactant is selected from: cocamidopropyl betaine (CAPB), cocamidopropyl sultaine, cocamidopropyl hydroxysultaine, lauramidopropyl betaine, lauramidopropyl sultaine, lauramidopropyl hydroxysultaine, oleamidopropyl betaine, oleamidopropyl sultaine, oleamidopropyl hydroxysultaine, tallowamideopropyl betaine, tallowamidopropyl sultaine, tallowamidopropyl hydroxysultaine, lauryl betaine, lauryl sultaine, lauryl hydroxysultaine, lauryldimethylamine oxide, and myristamine oxide.
1.49 Any of Compositions 1.42-1.47, wherein said cationic surfactant is selected from: cetylpyridinium chloride (CPC), cetrimonium bromide, benzalkonium chloride, benzethonium chloride (1-hexadecylcarbamoyl-ethyl)-trimethylammonium halide, (1-hexadecylcarbamoyl-2-phenyl-ethyl)-trimethylammonium halide, 1-hexadecylcarbamoyl-1,1-dimethyl-pyrrolidinium halide, and [2-(1H-indole-3-yl)-1-hexadecylcarbamoyl-ethyl)]-trimethylammonium halide, wherein said halide is optionally chloride, fluoride or bromide, or lauroyl arginine, ethyl lauroyl arginine ester hydrochloride, and disodium sebacoyl bis-lauramidolysine.
1.50 Any of Compositions 1.42-1.49, wherein said non-ionic surfactant is selected from: cocomonoethanolamide, cocodiethanolamide, laurylamidopropyl dimethylamine oxide, myristylamidopropyl dimethylamine oxide, and decyl glucoside.
1.51 Any of Compositions 1.42-1.50, wherein the oral care composition comprises an anionic surfactant and a zwitterionic surfactant.
1.52 Composition 1.51, wherein the oral care composition comprises sodium lauryl sulfate and cocamidopropyl betaine.
1.53 Composition 1.52, wherein the oral care composition comprises 0.1 to 5 wt. %, 1 to 5 wt. %, 2 to 4 wt. %, 1 to 3 wt. %, or to 3 wt. %, 1.5 to 2.5 wt. %, or 2 to 2.5 wt. %, or about 2 wt. %, of sodium lauryl sulfate, and 0.1 to 1 wt. %, 0.1 to 0.5 wt. %, or about 0.3 wt. %, of cocamidopropylbetaine, wherein the weight percentages are based on the total weight of the oral care composition.
1.54 Any preceding Composition, wherein the oral care composition further comprises an antioxidant, e.g., selected from butylated hydroxyanisole, butylated hydroxytoluene, vitamin A, carotenoids, vitamin E, flavonoids, polyphenols, ascorbic acid, and mixtures thereof.
1.55 Composition 1.54, wherein the antioxidant is butylated hydroxyanisole or butylated hydroxytoluene.
1.56 Composition 1.55, wherein the antioxidant is butylated hydroxytoluene.
1.57 Any of Compositions 1.54-1.57, wherein the oral care composition comprises any one or more antioxidants in an individual amount of 0.001 to 1 wt. %, e.g., 0.01 wt. % to 0.5 wt. %, 0.01 to 0.3 wt. %, 0.01 to 0.1 wt. %, 0.01 to 0.05 wt. %, or about 0.03 wt. %, based on the total weight of the oral care composition.
1.58 Any preceding Composition, wherein the oral care composition further comprises a polyphosphate or an organic cyclic polyphosphate, such as an alkali metal pyrophosphate, an alkali metal tripolyphosphate, an alkali metal tetraphosphate, an alkali metal hexametaphosphate, an alkali metal insoluble metaphosphate, an alkali metal phytic acid salt, or a mixture thereof.
1.59 Composition 1.58, wherein the oral care composition comprises a sodium or potassium pyrophosphate, a sodium or potassium tripolyphosphate, a sodium or potassium tetraphosphate, a sodium or potassium phytic acid salt, or a mixture thereof.
1.60 Composition 1.59, wherein the oral care composition comprises a tetra-alkali metal pyrophosphate, e.g., tetrasodium or tetrapotassium pyrophosphate.
1.61 Composition 1.60, wherein the oral care composition comprises a di-alkali metal pyrophosphate, e.g., disodium pyrophosphate or dipotassium pyrophosphate.
1.62 Composition 1.59, wherein the oral care composition comprises a tetra-alkali metal pyrophosphate and a di-alkali metal pyrophosphate, e.g., tetrasodium pyrophosphate (TSPP) and disodium pyrophosphate (DSPP).
1.63 Any of Compositions 1.58-1.62, wherein the oral care composition comprises from 0.1 to 5%, by weight, of polyphosphates, e.g., 0.5 to 5%, 1% to 5%, 2% to 5%, 3 to 5%, 4% to 5%, 1 to 4%, 2 to 4%, 3 to 4%, 1 to 3%, 2 to 3%, 1 to 2%, about 1.5%, about 2%, about 2.5%, by weight, based on the total weight of the oral care composition.
1.64 Composition 1.63, wherein the oral care composition comprises 1-5 wt. %, 1-4 wt. %, 1-3 wt. %, or 1-2 wt. %, of a tetra-alkali metal pyrophosphate and 0.1-1 wt. %, 0.2-1 wt. %, 0.25-1 wt. %, or 0.5-0.9 wt. % of a di-alkali metal pyrophosphate, e.g., about 1.3 wt. %, 1.5 wt. %, or 1.7 wt. % of tetrasodium pyrophosphate and about 0.4 wt. %, 0.5 wt. %, 0.6 wt. %, or 0.8 wt. % of disodium pyrophosphate, where the weight percentage is based on the total weight of the oral care composition.
1.65 Any preceding Composition, wherein the oral care composition further comprises a fluoride source.
1.66 Composition 1.65, wherein the fluoride source is selected from sodium fluoride, sodium monofluorophosphate, and stannous fluoride, or mixtures thereof.
1.67 Composition 1.66, wherein the fluoride source is sodium monofluorophosphate.
1.68 Any of Compositions 1.65-1.67, wherein the composition comprises the fluoride source (e.g., sodium monofluorophosphate) in an amount of 0.1 to 3 wt. %, e.g., 0.5 to 2 wt. %, or 0.5 to 1.5 wt. %, or 0.7 to 1.4 wt. %, or 0.75 to 1.2 wt. %, or about 0.75 wt. % or about 1.15 wt. %, based on the total weight of the oral care composition.
1.69 Any preceding Composition, wherein the composition further comprises one or more sweeteners, flavors, and/or colors.
1.70 Any preceding Composition, wherein the composition further comprises a blue dye or pigment, e.g., Blue 15 pigment (also known as CI 74160).
1.71 Composition 1.70, wherein the composition comprises the blue dye or pigment in an amount of 0.001 to 0.1% by weight of the oral care composition, e.g., 0.01 to 0.08%, or 0.03 to 0.07%, or about 0.05%, by weight, where the weight percentage is based on the total weight of the oral care composition.
1.72 Any preceding Composition, wherein the composition has a pH of about 4.5-7.5, e.g., 5.0-7.5, or 5.5-7.5, or 6-7.5, or 6.5-7.5, or 7-7.5. The composition may be formulated to specifically have certain pHs. For instance, in some embodiments, the composition has a pH of about 4.5 to about 8, about 5 to about 8, about 5.5 to about 8, about 6 to about 8, about 6.5 to about 8; from about 4.5 to about 7.5, about 5 to about 7.5, about 5.5 to about 7.5, about 6 to about 7.5, about 6.5 to about 7.5; from about 4.5 to about 7, about 5 to about 7, about 5.5 to about 7, about 6 to about 7, about 6.5 to about 7; from about 4.5 to about 6.5, about 5 to about 6.5, about 5.5 to about 6.5, about 6 to about 6.5; from about 4.5 to about 6, about 5 to about 6, about 5.5 to about 6; from about 4.5 to about 5.5, about 5 to about 5.5, or any range or subrange thereof. In at least one embodiment, the pH of the composition is about 5.5 to about 6 or 5.5 to 6.
1.73 Any preceding Composition, wherein the oral care composition comprises 1-6 wt. % hydrogen peroxide, 1-6% water, 12-18 wt. % of calcium pyrophosphate abrasive, 40-60 wt. % propylene glycol, 8-14 wt. % polyvinylpyrrolidone, 5-18 wt. % of a polyethylene glycol/polypropylene glycol random copolymer, 1-5 wt. % fumed silica, 1-5 wt. % of an anionic surfactant (e.g., sodium lauryl sulfate), 0.01-0.5 wt. % of an antioxidant (e.g., BHT); wherein the hydrogen peroxide is incorporated into the composition as an about 50 wt. % aqueous hydrogen peroxide solution.
1.74 Composition 1.73, wherein the oral care composition comprises about 1 wt. %, about 2 wt. %, about 3 wt. %, about 4 wt. %, about 5 wt. %, or about 6 wt. % hydrogen peroxide.
1.75 Composition 1, or any of 1.1-1.74, wherein the oral care composition comprises the following formula:

| Ingredient | Wt. % | |
|---|---|---|
| Propylene Glycol | 40-60% | (e.g., 45-55%) |
| Aqueous Hydrogen Peroxide | 1-10% | (e.g., 1-6%) |
| Solution (50 wt. % Hydrogen | | |
| peroxide) | | |
| PVP (e.g., cPVP) | 6-18% | (e.g., 8-14%) |
| Sodium Lauryl Sulfate | 1-3% | (e.g., 2%) |
| Fumed Silica | 1-6% | (e.g., 1.5-5.5%) |
| Calcium Pyrophosphate | 5-20% | (e.g., 8-18%) |
| Tetrasodium Pyrophosphate | 1-5% | (e.g., 1-2%) |
| (TSPP) | | |
| Disodium Pyrophosphate (DSPP) | 0.1-2% | (e.g., 0.25-1%) |
| Sodium Monofluorophosphate | 0.5-2% | (e.g., 0.75-1.2%) |
| PEG/PPG-116/66 Copolymer | 5-15% | (e.g., 7.5-10%) |
| Flavoring Agents | 0.1-4% | |
| Preservative(s) | 0-0.05% | (e.g., 0.02-0.03%) |

1.76 Any preceding Composition, wherein the weight ratio of polyvinylpyrrolidone to hydrogen peroxide in the oral care composition is about 9:1 to about 1.5:1, e.g., 5:1 to 1.5:1, or 4:1 to 1.5:1, or 3:1 to 1.5:1, or 2.5:1 to 1.5:1, or 2.25:1 to 1.5:1, or 2:1 to 1.5:1, or 1.8:1 to 1.5:1, or about 4.5:1, or about 3:1, or about 2.25:1, or about 1.8:1.
1.77 Any preceding Composition, wherein the composition is a dentifrice, e.g., a toothpaste or gel.
1.78 Any preceding Composition, wherein the oral care composition does not comprise polyethylene glycol.
1.79 Any preceding Composition, wherein the oral care composition does not comprise a poloxamer (e.g., an ethylene oxide-propylene oxide triblock copolymer).
1.80 Any preceding Composition, wherein the oral care composition does not comprise a polycarboxymethylene polymer (e.g., a polyacrylic acid or polyacrylate, such as a Carbopol).
1.81 Any preceding Composition, wherein the oral care composition does not comprise a povidone-iodine complex.
1.82 Any preceding Composition, wherein the oral care composition is a single-phase and single-component composition (i.e., not a dual-phase or dual-component composition).
1.83 Any preceding Composition, wherein the composition exhibits about 5% or less of hydrogen peroxide degradation when stored at a temperature of 40° C. for 1 month, e.g., about 4% or less, about 3% or less, about 2% or less, about 1% or less, or about 0.5% or less of hydrogen peroxide degradation (compared to the level of hydrogen peroxide present in the oral care composition when packaged).

In a second aspect, the present disclosure provides a method of making an aqueous abrasive oral care composition (e.g., Composition 1, or any of 1.1-1.83) comprising 1-10 wt. % hydrogen peroxide, comprising the step of incorporating a 40-80% aqueous solution of hydrogen peroxide (e.g., 50% aqueous hydrogen peroxide) into the oral care composition. In one embodiment, wherein the method provides a Composition 1, or any of 1.1-1.83, the method comprises the steps of: (a) combining the 50% aqueous hydrogen peroxide with the propylene glycol and mixing the same, (b) adding other ingredients other than the gelling agents (e.g., adding phosphate salts, fluoride sources, sweeteners and/or flavors) and mixing the same, (c) adding the gelling agents (e.g., PVP and/or fumed silica) and mixing the same, and (d) adding the abrasives (e.g., calcium pyrophosphate), surfactants (e.g., SLS), and mixing the same.

In another embodiment, the same steps (a) through (d) may be carried out, except that the aqueous hydrogen peroxide is added at step (b), step (c), or step (d). In some embodiments, the aqueous hydrogen peroxide is added as the last addition step of the method, or the second to last addition step of the method (e.g., wherein addition of the abrasive is the last addition step of the method). In some embodiments, the aqueous hydrogen peroxide and the PVP are not introduced in the same step. In some embodiments, the abrasive and the hydrogen peroxide are not introduced in the same step. In some embodiments, the fumed silica and the hydrogen peroxide are not introduced in the same step. In some embodiments, each of the aqueous hydrogen peroxide, the PVP, and the fumed silica, are added at different steps from each other.

In another embodiment, the method comprises the steps of (a) combining propylene glycol, antioxidant (e.g., BHT) and PEG/PPG random copolymer (e.g., PEG/PPG 116/66) and mixing same, (b) adding phosphates (TSPP and DSPP) and mixing same, (c) adding fluoride (e.g., MFP) and sweeteners and mixing same, (d) adding 50% aqueous hydrogen peroxide and mixing same, (e) adding anionic surfactant (e.g., SLS) and mixing same, (f) adding fumed silica and mixing same, (g) adding PVP and mixing same, (h) adding calcium pyrophosphate and mixing same, and (i) adding flavors and any other ingredients and mixing same. In some embodiments, steps (h) and (i) may be reversed in order.

In any of the preceding method of making embodiments, the hydrogen peroxide solution may be added to a vessel or intermediate composition having a temperature of about 30° C. or less, about 28° C. or less, about 26° C. or less, or about 24° C. or less.

According to another aspect, the present disclosure provides a method for using the oral care compositions disclosed herein. In some embodiments, where the oral care composition of the disclosure is a dentifrice (e.g., a toothpaste), the oral care composition may be applied to a user's teeth according to typical means for brushing teeth. In some embodiments, the present invention provides a method of whitening a tooth surface; cleaning an oral cavity surface; or treating, preventing or ameliorating a disease, disorder or condition of the oral cavity, comprising: applying an effective amount of an oral care composition to an oral cavity surface of a subject in need thereof. The disease, disorder, or condition for which the oral care composition treats, prevents, or ameliorates is selected from: gingivitis; periodontitis; excessive plaque and/or tartar build-up; caries; tooth decay; stained teeth (e.g., intrinsic or extrinsic stains); halitosis; erosion; sensitivity; inflammation; and a combination of two or more thereof.

The present disclosure further provides a kit comprising the aqueous abrasive oral care composition according to the disclosure (e.g., Composition 1, or any of 1.1-1.83). The kit may include a container and an oral care composition disposed in the container, and optionally instructions for using the kit. The oral care compositions can be packaged into containers or dispensers known in the art, via means conventional in the art. In some embodiments the compositions are packaged into tubes, metal, plastic or laminated, with either screw top or flip top caps. The container may be compatible with or contain a pump.

In another aspect, the present disclosure provides for the use of a 40-80% aqueous solution of hydrogen peroxide (e.g., 50% aqueous hydrogen peroxide) in the making of an aqueous abrasive oral care composition (e.g., Composition 1, or any of 1.1-1.83) comprising 1-10 wt. % hydrogen peroxide.

Common polyol humectants include propylene glycol, butylene glycol, hexylene glycol, pentylene glycol, 1,3-propanediol, diethylene glycol, dipropylene glycol, caprylyl glycol, and glycerin. The term “polyol humectant” refers to a polyhydroxylated small molecule, such as a polyhydroxylated C3-C6 compound. The term does not embrace polymers, such as polyethylene glycol, polypropylene glycol, or polyethylene glycol/polypropylene glycol copolymers. In some embodiments, some portion of the above recited amounts of propylene glycol may be substituted for by a different polyol humectant, provided that the composition comprises at least 20 wt. % of propylene glycol. Preferably, the compositions are free of glycerol because it has been found to be particularly destabilizing for aqueous hydrogen peroxide compositions. Preferably, the only polyol humectant in the composition is propylene glycol.

Polyethylene glycol (PEG) and polypropylene glycol (PPG) are both homopolymers comprises of repeating units derived from either ethylene oxide (EO) or propylene oxide (PO), respectively. PEG and PPG are distinct from ethylene oxide/propylene oxide copolymers (EO/PO copolymers), also known as PEG/PPG copolymers, which are heteropolymers made from both ethylene oxide and propylene oxide. PEG/PPG copolymers may be random copolymers, in which there is no order or structure to the combination of ethylene oxide and propylene oxide subunits in the polymer. They are defined primarily by the molar ratio of EO to PO and the molecular weight of the copolymers. These polymers include the Pluracare® and Pluraflo® lines of polymers marketed by BASF, such as Pluracare® L1120 (PEG/PPG 116/66). They are also known by corresponding INCI names, such as PEG/PPG 116/66, which refers to a 116/66 molar ratio of EO to PO in the copolymer.

Distinct from the random PEG/PPG copolymers are the triblock PEG/PPG copolymers, commonly referred to as poloxamers. Poloxamers have the following chemical structure:

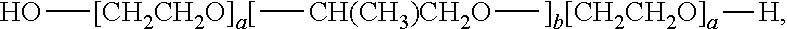

wherein a and b are integers, each typically between 10 and 200. Poloxamers are named according to common conventions based on their molecular weight and ethoxy content, and include poloxamer 407, poloxamer 338, poloxamer 237, poloxamer 188 and poloxamer 124. Pluronic® is the name of a line of poloxamer polymers manufactured by BASF. For example, Pluronic F-127 is poloxamer 407. The compositions of the present disclosure preferably do not comprise poloxamers.

Suitable 50% aqueous hydrogen peroxide solutions for use in the present invention include those products marketed under the trade names: PERSYNT 500 Super D, PERSYNT 500 Alkali and PERSYNT 500 Cosmetic by Evonik; PEROXAL 35 CG, PEROXAL 50 CG from Arkema; or INTEROX from Solvay.

In some aspects the oral care composition may comprise about 5 wt. % or less, optionally about 4 wt. % or less, optionally about 3 wt. % or less, optionally about 2 wt. % or less, or optionally about 1 wt. % or less, of a fatty compound. In some instances, the oral care composition is substantially free of fatty compounds or, optionally, is free of fatty compounds. The fatty compounds may be fatty alcohols, fatty esters, fatty ethers, fatty oils (e.g., hydrocarbon oils), derivatives thereof, and/or a combination of two or more thereof.

As noted above, certain embodiments of the oral care compositions have reduced amount or are free of fatty amphiphile. For instance, the oral care composition may have about 3 wt. % or less, about 2 wt. % or less, about 1 wt. % or less, about 0.5 wt. % or less, about 0.1 wt. % or less, or about 0.05 wt. % or less, of fatty amphiphiles. In certain embodiments, the oral care composition is substantially free of or free of fatty amphiphilcs.

As used herein, the term “aqueous abrasive oral care composition” means an oral care composition comprising an abrasive and comprising added water, preferably wherein the added water is solely derived from the aqueous hydrogen peroxide component of the composition.

As used herein, the term “added water” refers to water added to the composition as a discrete ingredient during the manufacture of the composition. This includes directly added water (e.g., distilled water, deionized water, or tap water), and the water of ingredients directly added as aqueous solutions (e.g., aqueous hydrogen peroxide, aqueous flavor solutions, aqueous polymer solutions). Added water does not include water of hydration in salts, or trapped or adsorbed water in hygroscopic solid ingredients (e.g., hydrated silica, fumed silica), or any water dissolved in polar solvents. Added water also does not include any water generated after manufacture of the composition by the slow decomposition of hydrogen peroxide.

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation.

Unless otherwise specified, all percentages and amounts expressed herein and elsewhere in the specification should be understood to refer to percentages by weight relative to the total composition. The amounts given are based on the active weight of the material unless specified otherwise.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of plus or minus 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Unless indicated otherwise, the abbreviation “wt. %” means percent by weight with respect to the entire oral care composition. Unless indicated otherwise, other references to percent or “%” also refer to percent by weight, except for aqueous hydrogen peroxide. It is understood that when referring to the percentage of hydrogen peroxide in an aqueous hydrogen peroxide solution used as an ingredient or component of the compositions disclosed herein, the percentage refers to the assay percentage recognized or certified by the manufacturer of the aqueous hydrogen peroxide solution. Commonly, hydrogen peroxide is sold in various grades, including 3%, 5%, 10%, 12%, 15%, 20%, 25%, 30%, 33%, 35%, 40%, 50%, 60%, 70%, 80%, 90%, and others from time to time. These are often not specified as either being weight percent (w/w), or volume percent (v/v), or some other ratio, and instead typically refer to the result of a quality control assay used to evaluate the concentration of the product (e.g., potassium permanganate titration assay). Nevertheless, these are generally believed to be approximately equal to weight percentage values. In addition, it is understood that these are not exact terms, but that a product meeting the required assay specification will typically be accepted over a narrow range (e.g., a 50% hydrogen peroxide solution could have from about 58 to 62% hydrogen peroxide by assay at the time of manufacture).

Such commercial aqueous hydrogen peroxide products also typically have a stabilizer present, such as tin-based stabilizing agents, often of a proprietary nature, typically in an amount of less than 1 wt. %. A particularly common stabilizer for commercial aqueous hydrogen peroxide is sodium stannate, such as at 50-500 mg/L concentration (about 0.08 wt. %), but lower concentrations are sufficient when it is combined with other synergistic stabilizers (e.g., phosphonic acids, pyrophosphates), down to as little as 1 mg/L or less.

As used herein the term “metal ion chelating agent” refers to agents which are metal ion chelating agents used for the stabilization of hydrogen peroxide against decomposition. The most important ions of interests in this regard are iron and copper.

It is understood that while the compositions of the present disclosure are preferably not formulated with a metal ion chelating agent, i.e., preferably no metal chelating agent is added as an ingredient or component during manufacture of the compositions, the compositions may have a trace amount of a metal chelating agent if one was included as a stabilizing agent in the aqueous hydrogen peroxide used as a component of the composition. For example, a stannate salt or complex derived from the aqueous hydrogen peroxide component might be present in the oral care composition in an amount of less than 0.1%, preferably less than 0.05%, or less than 0.01%, or less than 0.005%, or less than 0.001%, or less than 0.0005%, or less than 0.0001%, by weight of the composition. Therefore, in general, a composition according to the present disclosure may have not more than 0.1 wt. % of metal ion chelating agents, such as metal stannate salts and EDTA or its salts, preferably less than 0.05%, or less than 0.01%, or less than 0.005%, or less than 0.001%, or less than 0.0005%, or less than 0.0001%, by weight of the composition. Preferably the compositions have less than 0.01% by weight of such agents.

As used herein, “high cleaning silica” can refer to silica having a pellicle cleaning ratio (PCR) of greater than 85 when tested at 20% loading as is accepted in the art as high cleaning silica. Typically, high cleaning silica also has a mean particle size d₅₀ of from 5 to 15 μm and an oil absorption of from 40 to 120 cm³/100 g silica. Examples of high cleaning silica include silica marketed under the brand name ZEODENT® 105 from Evonik.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the oral care compositions of the instant disclosure can be free or essentially free of all components and elements positively recited throughout the instant disclosure. In some instances, the oral care compositions of the present disclosure may be substantially free of non-incidental amounts of the ingredient(s) or compound(s) described herein. A non-incidental amount of an ingredient or compound is the amount of that ingredient or compound that is added into the oral care composition by itself. For example, an oral care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the oral care composition includes both components (or the composition includes more than two components that overlap), an overlapping compound does not represent more than one component. For example, certain compounds may be characterized as both an emulsifier and a surfactant. If a particular oral care composition includes both an emulsifier and a surfactant, a compound that may be characterized as both an emulsifier and a surfactant will serve only as either an emulsifier or a surfactant—not both.

Aspects of the present invention relate to oral care compositions and methods of making and using the same. The inventors discovered that oral care compositions having certain ingredients in particular weight ratios surprisingly have an enhanced stability for hydrogen peroxide, even in the presence of water and at a pH of 4.5 or greater. For example, the oral care compositions disclosed herein may exhibit about 5% or less of the hydrogen peroxide degradation when stored at a temperature of 40° C. for 1 month. Preferably, about 4% or less, about 3% or less, about 2% or less, about 1% or less, or about 0.5% or less of the hydrogen peroxide present in the oral care composition degrades when stored at a temperature of 40° C. for 1 month.

In some embodiments, the oral care composition may be formulated to have a viscosity from about 100,000 to about 1,000,000 centipoise (cP), about 50,000 to 250,000 cP, about 70,000 to 225,000 cP, about 100,000 to 200,000 cP, including all values in between these ranges, at room temperature using Brookfield viscometer and a spindle no. 3. For example, the oral care composition may have a viscosity of about 100,000 to about 1,000,000 cP, about 100,000 to about 900,000 cP, about 100,000 to about 800,000 cP, about 100,000 to about 700,000 cP, about 100,000 to about 600,000 cP, about 100,000 to about 500,000 cP, about 100,000 to about 400,000 cP, about 100,000 to about 300,000 cP, about 100,000 to about 250,000 cP, about 100,000 to about 200,000 cP; from about 150,000 to about 1,000,000 cP, about 150,000 to about 700,000 cP, about 150,000 to about 600,000 cP, about 150,000 to about 500,000 cP, about 150,000 to about 400,000 cP, about 150,000 to about 300,000 cP, about 150,000 to about 250,000 cP, about 150,000 to about 200,000 cP; from about 200,000 to about 1,000,000 cP, about 200,000 to about 700,000 cP, about 200,000 to about 600,000 cP, about 200,000 to about 500,000 cP, about 200,000 to about 400,000 cP, about 200,000 to about 300,000 cP, about 200,000 to about 250,000 cP; from about 250,000 to about 1,000,000 cP, about 250,000 to about 700,000 cP, about 250,000 to about 600,000 cP, about 250,000 to about 500,000 cP, about 250,000 to about 400,000 cP, about 250,000 to about 300,000 cP; from about 350,000 to about 1,000,000 cP, about 350,000 to about 800,000 cP, about 350,000 to about 700,000 cP, about 350,000 to about 600,000 cP, about 350,000 to about 500,000 cP; from about 450,000 to about 1,000,000 cP, about 450,000 to about 900,000 cP, about 450,000 to about 800,000 cP, about 450,000 to about 700,000 cP, about 450,000 to about 600,000 cP; from about 550,000 to about 1,000,000 cP, about 550,000 to about 900,000 cP, about 550,000 to about 800,000 cP, about 550,000 to about 700,000 cP; from about 650,000 to about 1,000,000 cP, about 650,000 to about 900,000 cP, about 650,000 to about 800,000 cP, including all values in between these ranges, at room temperature using Brookfield viscometer and a spindle no. 3, e.g., for 1 minute.

The oral care compositions of the disclosure may be in the form of, e.g., a dentifrice (e.g., a toothpaste or toothpowder or oral gel). Suitable components, such as those listed below, may be included or excluded from the formulations for the oral care compositions depending on the specific combination of other ingredients and the form of the oral care compositions. In a further aspect the oral care compositions of the disclosure be in a form selected from the following: dentifrice (e.g., toothpaste), a topical oral gel, or tooth powder.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility.

In some embodiments, the oral care compositions as disclosed herein are used by humans. Examples of oral care compositions include, but are not limited to, toothpaste or a dentifrice, a mouthwash or a mouth rinse, a topical oral gel, a denture cleanser, sprays, toothpaste powders, tablets, mousse, foam, chewing gums and the like.

As used herein, the term “dentifrice” means a paste, gel, or semi-solid formulation, unless otherwise specified. The dentifrice composition can be in any desired form, e.g., deep striped, surface striped, multi-layered, having the gel surrounding the paste, or any combination thereof. Alternatively, in one aspect, the oral composition may be dual phase dispensed from a separated compartment dispenser.

The oral care compositions typically comprise one or more thickening agent, e.g., in an amount from about 6 to about 18 wt. %, about 6 to about 16 wt. %, about 6 to about 14 wt. %, about 6 to about 12 wt. %, about 6 to about 11 wt. %, about 6 to about 10 wt. %, about 6 to about 9 wt. %, about 6 to about 8 wt. %; from about 7 to about 18 wt. %, about 7 to about 16 wt. %, about 7 to about 14 wt. %, about 7 to about 12 wt. %, about 7 to about 11 wt. %, about 7 to about 10 wt. %, about 7 to about 9 wt. %; from about 8 to about 18 wt. %, about 8 to about 16 wt. %, about 8 to about 14 wt. %, about 8 to about 12 wt. %, about 8 to about 11 wt. %, about 8 to about 10 wt. %, about 8 to about 9 wt. %; from about 9 to about 18 wt. %, about 9 to about 16 wt. %, about 9 to about 14 wt. %, about 9 to about 12 wt. %, about 9 to about 11 wt. %, about 9 to about 10 wt. %; from about 10 to about 18 wt. %, about 10 to about 16 wt. %, about 10 to about 14 wt. %, about 10 to about 12 wt. %; from about 12 to about 18 wt. %, about 12 to about 16 wt. %, about 12 to about 14 wt. %; about 16 to about 18 wt. %, or any range or subrange thereof, based on the total weight of the oral care composition.

The oral care composition may comprise a thickening system formed from a plurality of thickening agents, such as two thickening agents, three thickening agents, four thickening agents, five thickening agents, or any range or subrange formed therefrom. In some embodiments, the thickening system comprises two or more thickening agents, three or more thickening agents, four or more thickening agents, or five or more thickening agents.

Preferably, the thickening system comprises polyvinyl pyrrolidone. Polyvinylpyrrolidone refers to a polymer containing vinylpyrrolidone (e.g., N-vinylpyrrolidone, N-vinyl-2-pyrrolidione, and N-vinyl-2-pyrrolidinone) as a monomeric unit. The monomeric unit may include a polar imide group, four non-polar methylene groups, and a non-polar methane group. The polyvinyl pyrrolidone may have an average molecular weight in the range 5,000 to 100,000, preferably in the range 5,000 to 50,000. Polyvinylpyrrolidones that have average molecular weights of 10,000, 30,000 and 40,000 may be commercially available from Sigma Chemical Co., GAF Corporation and Sigma Chemical Co. In some embodiments, the polyvinylpyrrolidone is crosslinked polyvinyl pyrrolidone (crosslinked PVP or cPVP).

The polyvinylpyrrolidone may be present in the oral care compositions in an amount from about 6 to about 18 wt. %, about 6 to about 16 wt. %, about 6 to about 14 wt. %, about 6 to about 12 wt. %, about 6 to about 11 wt. %, about 6 to about 10 wt. %, about 6 to about 9 wt. %, about 6 to about 8 wt. %; from about 7 to about 18 wt. %, about 7 to about 16 wt. %, about 7 to about 14 wt. %, about 7 to about 12 wt. %, about 7 to about 11 wt. %, about 7 to about 10 wt. %, about 7 to about 9 wt. %; from about 8 to about 18 wt. %, about 8 to about 16 wt. %, about 8 to about 14 wt. %, about 8 to about 12 wt. %, about 8 to about 11 wt. %, about 8 to about 10 wt. %, about 8 to about 9 wt. %; from about 9 to about 18 wt. %, about 9 to about 16 wt. %, about 9 to about 14 wt. %, about 9 to about 12 wt. %, about 9 to about 11 wt. %, about 9 to about 10 wt. %; from about 10 to about 18 wt. %, about 10 to about 16 wt. %, about 10 to about 14 wt. %, about 10 to about 12 wt. %; from about 12 to about 18 wt. %, about 12 to about 16 wt. %, about 12 to about 14 wt. %; about 16 to about 18 wt. %, or any range or subrange thereof, based on the total weight of the oral care composition.

In certain preferred embodiments, the thickening system further comprises a silica thickener, such as fumed silica. The silica thickener (e.g., fumed silica) may be present in the oral care composition in an amount from about 1 to about 5 wt. %, about 1 to about 4 wt. %, about 1 to about 3.5 wt. %; from about 2 to about 5 wt. %, about 2 to about 4 wt. %, about 2 to about 3.5 wt. %; from about 3 to about 5 wt. %, about 3 to about 4 wt. %, about 3 to about 3.5 wt. %, or any range or subrange thereof, based on the total weight of the oral care composition. Without being limited to any particular theory, it believed that certain embodiments of the oral care composition having particular thickening systems containing PVP and about 2 wt. % or more silica thickener (e.g., fumed silica) in certain ratios and certain preferred pH values may achieve enhanced flavor profiles and/or textures. In at least one embodiment, the amount of fumed silica present in the oral care composition is about 2 to about 3.5 wt. %, 2.5 to about 3.5 wt. % or about 3 to about 3.5 wt. %, based on the total weight of the oral care composition.

The oral care composition may comprise polyvinylpyrrolidone and silica thickener in certain preferred ratios. In some embodiments, the oral care composition has a weight ratio of polyvinylpyrrolidone to silica thickeners of about 5:1 to about 1:1, about 4:1 to about 1:1, about 3:1 to about 1:1, about 2:1 to about 1:1; from about 5:1 to about 2:1, about 4:1 to about 2:1, about 3:1 to about 2:1; from about 5:1 to about 3:1, about 4:1 to about 3:1, or any range or subrange thereof. In certain embodiments, the one or more thickening agents of the oral care composition consist of one or more thickening agent selected from (e.g., cross-linked polyvinylpyrrolidone) and/or silica thickeners (e.g., fumed silica).

In some aspects, the oral care compositions of the disclosure may include additional and/or optional thickeners other than polyvinyl pyrrolidone and silica thickener. Illustrative additional or optional thickeners other than polyvinyl pyrrolidone may be or include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX®, which is commercially available from The Dow Chemical Company of Midland, Mich.), cellulosic polymers, hydroxyethyl cellulose, carboxymethylcellulose, and salts thereof (e.g., CMC sodium), natural gums (e.g., karaya, xanthan, gum arabic, and tragacanth), colloidal magnesium aluminum silicate, and the like, and mixtures or combinations of two or more thereof. Further additional thickening agents include a polyacrylate, a polymethacrylate, a polyitaconate, an acrylamide, 2-acrylamido-2-methylpropane sulfonic acid (AMPS), and fatty amphiphiles. In certain embodiments, one or more of the additional or optional thickening agents disclosed herein are excluded from the oral care composition.

The oral care compositions may include any of the following additional ingredients in an amount of from about 0.01 to about 15 wt. %, based on the total weight of the oral care composition. In some instances, the amount of additional ingredients present in the oral care composition is from about 0.01 to about 12.5 wt. %, about 0.01 to about 10 wt. %, about 0.01 to about 8 wt. %, about 0.01 to about 6 wt. %, about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 12.5 wt. %, about 0.1 to about 10 wt. %, about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 12.5 wt. %, about 0.5 to about 10 wt. %, about 0.1 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 0.75 to about 12.5 wt. %, about 0.75 to about 10 wt. %, about 0.75 to about 8 wt. %, about 0.75 to about 6 wt. %, about 0.75 to about 5 wt. %, about 0.75 to about 4 wt. %, about 0.75 to about 3 wt. %, about 0.75 to about 2 wt. %, about 0.75 to about 1 wt. %; about 1 to about 12.5 wt. %, about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 12.5 wt. %, about 3 to about 10 wt. %, about 3 to about 8 wt. %, about 3 to about 6 wt. %, about 3 to about 5 wt. %, or about 3 to about 4 wt. %, including any range or subrange therebetween, based on the total weight of the oral care composition.

Anionic surfactants may be selected from water-soluble, water-miscible salts of alkyl sulfate, such as those having from 8 to 20 carbon atoms in the alkyl radical (e.g., sodium alkyl sulfate) and the water-soluble or water-miscible salts of sulfonated monoglycerides of fatty acids having from 8 to 20 carbon atoms. Examples of suitable anionic surfactants include sodium lauryl sulfate, sodium lauryl sarcosinate, sodium cocoyl methyl taurate, sodium monoglyceride sulfate, sodium cetaryl sulfate, potassium cocoyl glycinate, sodium lauryl phosphate, sodium lauryl lactylate, sodium lauryl sulfoacetate, sodium lauryl glutamate, sodium lauryl isethionate, sodium laureth carboxylate, sodium dodecyl benzenesulfonate, and combinations thereof. In one embodiment, sodium lauryl sulfate is a preferred swelling surfactant. Additional anionic surfactants are disclosed in U.S. Pat. No. 3,959,458, which is incorporated herein in its entirety for all purposes.

Further anionic surfactants of mention include long chain alkyl (C₆-C₂₂) materials, such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, for example, ethoxylated, although alkoxylation is not required.

The fluoride ion source may be a fluorine-containing compound. Suitable ionic fluorine-containing compounds include fluoride salts, such as amine fluorides, alkali metal fluoride salts (e.g., sodium fluoride), and monofluorophosphate salts, such as alkali metal monofluorophosphate salts (e.g., sodium monofluorophosphate). In some embodiments, the fluoride ion source may be chosen from fluoride salts, such as sodium fluoride, potassium fluoride, calcium fluoride, zinc fluoride, stannous fluoride, zinc ammonium fluoride, sodium monofluorophosphate, potassium monofluorophosphate, laurylamine hydrofluoride, and combinations of two or more thereof. In at least one embodiment, the fluoride ion source may comprise or consist of sodium monofluorophosphate.

In some embodiments, the oral care composition of the disclosure may include a buffering system. The buffering system may include one or more buffering agents. While in some instances, the buffering system comprises pH adjusters, in other instances the buffering system only consists of buffering agents. The oral care composition may, additionally or alternatively, comprise one or more pH adjusters to increase or decrease the overall pH of the oral care composition. For example, one or more acids may be included to decrease the pH of the oral care composition. Examples of suitable acids for decreasing the pH of the oral care composition include, but are not limited to, citric acid, acetic acid, and the like. The oral care composition may include one or more bases, such as sodium hydroxide, potassium hydroxide and the like, to increase the pH of the oral care composition. Additional or alternative acids and bases that are suitable for adjusting the pH of the oral care composition are readily known to one of ordinary skill in the art.

The amount of the pH adjuster in the oral care composition may be based on the desired pH of the final oral care composition and/or product. For example, the total amount of the pH adjuster may range from about 0.05 to about 20 wt. %, based on the total weight of the oral care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the oral care composition.

In some embodiments, the oral care composition may comprise a nonionic surfactant. Nonionic surfactants useful herein may include those compounds produced by the condensation of alkylene oxide groups (hydrophilic in nature) with an organic hydrophobic compound, which may be aliphatic or alkyl-aromatic in nature. Non-limiting examples of suitable nonionic surfactants include polyoxyethylene sorbitan esters (sold under the trade name Tweens), polyoxyl 40 hydrogenated castor oil, fatty alcohol ethoxylates, ethylene oxide condensates of aliphatic alcohols, long chain tertiary amine oxides, long chain tertiary phosphine oxides, lauryl glucoside (sold under the trade name Plantaren 1200 UP) and long chain dialkyl sulfoxides. Suitable nonionic surfactants with a HLB of 7 or more include sucrose laurate, sucrose cocoate, sucrose stearate; Steareth 20, 21, or 100, and PEG 20 Sorbitan Monostearate (commercially available as Tween 60). In at least one embodiment, the nonionic surfactants are chosen from polyethoxylated sorbitol esters, in particular polyethoxylated sorbitol monoesters; polycondensates of ethylene oxide and propylene oxide (poloxamers), for instance the products marketed under the trade name PLURONIC by BASF-Wyandotte; condensates of propylene glycol; polyethoxylated hydrogenated castor oil, for instance, cremophors; and sorbitan fatty esters.

Non-limiting examples of amphoteric surfactants include, for example, long chain imidazoline derivatives such as the product marketed under the trade name ‘Miranol C2M’ by Miranol; long chain alkyl betaines, such as the product marketed under the tradename ‘Empigen BB’ by Albright+Wilson, and long chain alkyl amidoalkyl betaines, such as cocamidopropylbetaine, and mixtures thereof.

In still a further aspect, the oral care composition of the disclosure may comprise amphoteric surfactants chosen from derivatives of aliphatic secondary and tertiary amines, in which the aliphatic radical can be a straight chain or branched and wherein one of the aliphatic substituents contains from about 8 to about 18 carbon atoms and one contains an anionic water-solubilizing group, such as carboxylate, sulfonate, sulfate, phosphate, or phosphonate. Other exemplary amphoteric surfactants are betaines, such as cocamidopropyl betaine, lauryl dimethyl betaine (sold under the trade name Macat LB), cetyl dimethyl betaine, and cocoamphodiacetate. Additional amphoteric surfactants and nonionic surfactants can be found in U.S. Pat. No. 4,051,234, which is incorporated herein in its entirety for all purposes.

Examples of cationic surfactants that may be present in the oral care composition include cetyl pyridinium chloride, cocamidopropyl PG dimonium chloride phosphate (Phospholipid CDM), myristylamidopropyl PG dimonium chloride phosphate (Phospholipid PTM), stearamidopropyl PG dimonium chloride phosphate (Phospholipid SV), steapyrium chloride (Catemol WPC), and other suitable cationic materials. The cationic surfactants may be D,L-2-pyrrolidone-5-carbo-xylic acid salt of ethyl-N-cocoyl-L-arginate, marketed under the trade name CAE by Ajinomoto Co. Inc.

The oral care composition may further comprise one or more colorants. The colorants may be a pigment, a dye, or mixtures thereof. Non-limiting examples of pigments include titanium dioxide, zinc oxide, kaolin, mica etc. Non-limiting examples of dyes include food dyes suitable for food, drug and cosmetic applications, and mixtures thereof. Some color agents (colorants) are known as FD&C dyes. In some embodiments, the colorants may be present in an amount ranging from about 0.0001% wt. % to about 0.4% wt. %, including all percentages and subranges therebetween, based on the total weight of the oral care composition. In further embodiments, the colorants may be present in an amount ranging from about 0.0001% wt. % to about 4% wt. %, including all percentages and subranges therebetween, based on the total weight of the oral care composition.

Examples of flavoring agents (flavors and/or flavoring materials) include: menthol; carvone; anethole; methyl salicylate; and the oils of spearmint, peppermint, wintergreen, sassafras, clove, sage, eucalyptus, marjoram, cinnamon, lemon, lime, grapefruit, kumquat, tangerine, and orange. Examples of sweeteners (sweetening agents) include sucrose, lactose, maltose, sorbitol, xylitol, sodium cyclamate, perillartine, L-aspartyl-L-phenylalanine methyl ester (aspartame), and saccharine.

In some aspects, the oral care compositions of the disclosure do not include any high cleaning silica. Further, in some aspects, the oral care composition of the disclosure does not include any high cleaning silica and demonstrates increased hydrogen peroxide stability (e.g., as measured by active oxygen levels) relative to a control formulation that comprises high cleaning silica.

In some embodiments, while the PVP and HP are not complexed or bound when added to the oral care composition, they may form a complex in situ after the addition to the oral care composition (e.g., a toothpaste). In this aspect, the PVP and HP are not bound or complexed together prior to their addition to the oral care composition (e.g., they are not added as a preformed complex).

Embodiments of the present invention will now be further described by way of the following, non-limiting, examples.

### EXAMPLES

### Example 1

Five non-limiting, example oral care compositions (Example Compositions A-E) are prepared in accordance with the formulations shown in Table 1, below. Two comparative oral care compositions (Comparative Compositions 1 and 2) were prepared using a pre-formed commercially obtained PVP-hydrogen peroxide complex. The formulations for the two comparative compositions are shown in Table 2, below. Each of Example Compositions A-E and Comparative Compositions 1 and 2 are prepared in the form of a toothpaste composition:

TABLE 1

| | Ex. A | Ex. B | Ex. C | Ex. D | Ex. E |
|---|---|---|---|---|---|
| Ingredient | Wt. % | | | | |
| Propylene Glycol | qs to 100 | qs to 100 | qs to 100 | qs to 100 | qs to100. |
| Aqueous Hydrogen Peroxide | 0.2 | 2 | 4 | 6 | 8 |
| Solution (50 wt. % Hydrogen | | | | | |
| peroxide) | | | | | |
| cPVP | 3.5 | 6 | 8 | 13 | 10 |
| Sodium Lauryl Sulfate | 2 | 2 | 2 | 2 | 2 |
| Fumed Silica | | 2.7-3.5 | 2.7-3.5 | 2.7-3.5 | 2.7-3.5 |
| Sodium Stearate | 2.5 | | | | |
| Calcium Pyrophosphate | 20 | 15 | 15 | 15 | 15 |
| Tetrasodium Pyrophosphate | 1-2 | 1-2 | 1-2 | 1-2 | 1-2 |
| (TSPP) | | | | | |
| Disodium Pyrophosphate (DSPP) | 0.25-1 | 0.25-1 | 0.25-1 | 0.25-1 | 0.25-1 |
| Sodium Monofluorophosphate | 1.1 | 0.76 | 0.76 | 0.76 | 0.76 |
| PEG/PPG-116/66 Copolymer | 7.5 | 10 | 10 | 10 | 10 |
| Flavoring Agents and Sweeteners | 3.1 | 3.1 | 3.1 | 3.1 | 3.1 |
| (Flavoring agents, sodium | | | | | |
| saccharin, and sucralose) | | | | | |
| Preservative(s) | | 0.03 | 0.03 | 0.03 | 0.03 |
| HP CONCENTRATION | 0.1 | 1 | 2 | 3 | 4 |

TABLE 2

| | |
|---|---|
| Comp. 1 | Comp. 2 |

| | |
|---|---|
| Ingredient | Wt. % |

| | | |
|---|---|---|
| Propylene Glycol | qs to 100 | qs to 100 |
| cPVP-HP | 16.5 | 22 |
| Sodium Lauryl Sulfate | 1.8-2.5 | 1.8-2.5 |
| Fumed Silica | 0.4 | 1.5 |
| Calcium Pyrophosphate | 15 | 15 |
| Tetrasodium Pyrophosphate | 1-1.8 | 1-1.8 |
| (TSPP) | | |
| Sodium Acid Pyrophosphate | 0.3-0.7 | 0.3-0.7 |
| Sodium Monofluorophosphate | 0.6-0.9 | 0.6-0.9 |
| PEG/PPG Copolymer | 7.5 | 7.5 |
| Flavoring Agents and Sweeteners | 3.3 | 3 |
| (Flavoring agents, sodium | | |
| saccharin, and sucralose) | | |
| Preservative(s) | 0.01-0.1 | 0.01-0.1 |
| HP CONCENTRATION | 3 | 4 |

The oral care compositions are prepared by adding 50% aqueous hydrogen peroxide to a tank followed by the propylene glycol. Next, the pyrophosphate salts, fluoride source, and sweeteners are added to the tank. After mixing, the mixture is transferred to the main mixing tank. Any gelling agents are added (PVP, fumed silica, PEG/PPG copolymer), and after thorough mixing, the remaining ingredients are added (abrasive, surfactants, flavors, etc.). After mixing, the product is dispensed into tubes.

### Example 2

Example Compositions B and C are studied under accelerated aging conditions to evaluate the stability of the hydrogen peroxide contained therein, by storing at a temperature of 40° C. for 4 weeks. The hydrogen peroxide concentration in the compositions is evaluated after 1 week, after 2 weeks, after 3 weeks, and after 4 weeks.

Example Compositions Formulas B and C were both determined to be stable, in that the hydrogen peroxide concentration exhibited only a small drop after 4-6 weeks of accelerated aging conditions, as shown in Table 2, below. For both formulas, only a 5% drop in the total HP concentration was observed.

TABLE 2

| | HP Concentration |
|---|---|
| | after 40° C. aging |
| Example Composition B (1% HP) | 0.95% after 4 weeks |
| Example Composition C (2% HP) | 1.9% after 6 weeks |

### Example 3

To evaluate the effect of adding abrasive silica on stability, formulas containing 4% hydrogen peroxide according to the Example Composition E (as shown in Example 1) are prepared, unmodified (Example Composition E) or modified to include either 5% or 10% high cleaning silica (Compositions F and G, respectively). The oral care compositions are aged under accelerated aging conditions at a temperature of 60° C. for 1 week. The remaining active oxygen levels are determined and found to be as follows (expressed as a percentage of the theoretical initial active oxygen content):

TABLE 3

| | | Percentage of | |
|---|---|---|---|
| | | Active Oxygen | |
| | | after 1 week | |
| Composition | | at 60° C. | |
| E | 4 wt. % HP, no high cleaning silica | 95.29 | wt. % |
| F | 4 wt. % HP, 5 wt. % high cleaning silica | 14.46 | wt. % |
| G | 4 wt. % HP, 10 wt. % high cleaning silica | 6.34 | wt. % |

*The percentage of active oxygen is relative to the initial amount of active oxygen.

The data demonstrates that a small amount of high cleaning silica is associated with a substantial loss of hydrogen peroxide stability in the formula. Therefore, to achieve good peroxide stability, it is essential to have a formula backbone with high purity material that is compatible with hydrogen peroxide, for example, not including high-cleaning silica or similar abrasives (or less than 5 wt. % thereof).

### Example 4

The stability, whitening efficacy, flavor, and texture are evaluated for formulations containing a 50 wt. % hydrogen peroxide aqueous solution. To assess the stability of the hydrogen peroxide, Example Compositions B-E were evaluated in comparison to comparative compositions that included a pre-formed commercially obtained PVP-hydrogen peroxide complex instead of the 50 wt. % hydrogen peroxide aqueous solution of Example Compositions B-E in an amount that delivered the equivalent amount of hydrogen peroxide and with minor modifications including calcium pyrophosphate abrasive (15 wt. %), PVP (0-3.75 wt. %) and fumed silica (0.4-3.75 wt. %) as thickeners, PEG/PPG 116/66 (5-10 wt. %), a combination of tetrasodium pyrophosphate and disodium pyrophosphate as anti-tartar agents (1.5-2 wt. %), sodium monofluorophosphate (0.76 wt. %), and a mixture of propylene glycol, glycerin, polyethylene glycol, and/or poloxamer L35 as humectants (net 45-65 wt. %). The comparative compositions are anhydrous, whereas Example Compositions B, C, D, and E have from 0.1 to 4 wt. % water from the 50 wt. % aqueous hydrogen peroxide solution.

### Stability

TABLE 4

| | 40° C. | | 40° C. | | 40° C. | |
|---|---|---|---|---|---|---|
| | 1 month | | 2 months | | 3 months | |
| | | Comparative | | Comparative | | Comparative |
| | | Composition | | Composition | | Composition |
| | | with | | with | | with |
| | | Commercially | | Commercially | | Commercially |
| | 50 wt. % | obtained | 50 wt. % | obtained | 50 wt. % | obtained |
| HP | Aq. HP | pre-formed | Aq. HP | pre-formed | Aq. HP | pre-formed |
| Conc. | formula | cPVP-HP | formula | cPVP-HP | formula | cPVP-HP |
| 1 wt. % | 1.0 wt. % | 1.03 wt. % | 0.96 wt. % | 0.92 wt. % | 0.94 wt. % | 0.89 wt. % |
| HP | (Ex. B) | | (Ex. B) | | (Ex. B) | |
| 2 wt. % | 2.0 wt. % | 1.90 wt. % | 2.0 wt. % | 1.90 wt. % | 1.9 wt. % | 1.80 wt. % |
| HP | (Ex. C) | | (Ex. C) | | (Ex. C) | |
| 3 wt. % | 3.1 wt. % | 2.9 wt. % | 3.0 wt. % | 2.9 wt. % | 2.8 wt. % | 2.8 wt. % |
| HP | (Ex. D) | | (Ex. D) | | (Ex. D) | |
| 4 wt. % | 4.1 wt. % | 4.1 wt. % | 4.0 wt. % | 4.1 wt. % | 3.8 wt. % | 3.9 wt. % |
| HP | (Ex. E) | | (Ex. E) | | (Ex. E) | |

The compositions are subjected to aging studies at a temperature of 40° C. over the course of one, two, and three months. As shown in Table 4, despite the presence of water, the Example Compositions B-E have comparable stability to the anhydrous formulas prepared using cPVP-HP complex. This trend is maintained over the course of three months.

### Whitening Efficacy

The whitening efficacy is evaluated using an in-vitro bovine enamel procedure according to standard procedures. Briefly, the heads of soft toothbrushes are cut from the handles and mounted for use on a brushing machine. Bovine teeth are mounted and stained with coffee and tea. For each formula sample, a 1:1 w/w slurry is prepared with deionized water. The slurry is poured over each tray and brushing is immediately started on the teeth. The teeth are brushed for 2 minutes with 250 grams of pressure applied. The brushing machine is set to 120 strokes per minute. After 2 minutes, the brushing is stopped, the slurry is removed, and the teeth are rinsed with deionized water then dried. The brushing treatment is repeated a total of 14 times to model twice daily use of each product for 7 days.

Software from Medical High Technology (MHT) is used to measure the L*, a*, and b* values for each tooth before and after treatment. The L*, a*, and b* values are used to calculate the change in the whiteness index for each tooth after 14 treatments as compared to baseline. The Whiteness index is reported as ΔW*, wherein:

$${W}^{*} = {( {a}^{* 2} + {b}^{* 2} + {( {L}^{*} - 100 )}^{2} )}^{1 / 2}$$

$$Δ {W}^{*} = {W}^{*} treated - {W}^{*} baseline$$

The absolute value of ΔW* is reported. It should be noted that the more positive the value of ΔW*, the closer the tooth color is to pure white.

The results show that substantially the same degree of whitening is achieved for each pair of tested formulas having the same net hydrogen peroxide concentration:

TABLE 5

| | Mean ΔW* after 14 treatments | |
|---|---|---|
| HP Conc. | 50% Aq. HP formula | Commercial cPVP-HP formula |
| 1% HP | 6.1 (Ex. B) | 6.1 |
| 2% HP | 8.0 (Ex. C) | 9.3 |
| 3% HP | 6.9 (Ex. D) | 6.9 |
| 4% HP | 10.1 | 10.1 |

The results show that toothpaste formulas that incorporate 50% aqueous hydrogen peroxide solution have equivalent whitening efficacy as commercial formulas that incorporate PVP-HP complex.

A 5% hydrogen peroxide formula substantially similar to Formula E, and a 5% hydrogen peroxide formula based on PVP-HP complex, are also prepared. Similar results are obtained in a follow-on study comparing these formulas with 5% HP. After 14 treatments, the mean ΔW* for the PVP-HP formula is found to be 9.9, and for the aqueous hydrogen peroxide formula it is found to be 10.4.

### Flavor and Texture Evaluation

Additionally, consumer testing is performed to evaluate the flavor of Example Compositions D and E. The consumer studies are done in a monadic manner, meaning each consumer only uses one type of toothpaste. For each study, 200 individuals (70% female and 30% male) are recruited and sent the product for usage (twice a day for 14 days). At the end of the study, a questionnaire is sent to each consumer for feedback.

The results of the consumer test showed that Example Composition D unexpectedly achieved about 20% higher number of individuals who indicated a strong preference for the flavor profile of Example Composition D relative to the number of individuals who indicated a strong preference for the flavor profile of Comparative Composition 1. Example Composition E unexpectedly achieved about 25% higher number of individuals who indicated a strong preference for the flavor profile of Example Composition E relative to the number of individuals who indicated a strong preference for the flavor profile of Comparative Composition 2.

The consumer at-home testing also evaluated the texture of Example Composition E in comparison to Comparative Composition 2. Example Composition E unexpectedly achieved an about 24% higher number of individuals who indicated a strong preference for the texture of Example Composition E relative to the number of individuals who indicated a strong preference for the texture of Comparative Composition 2.

The results of the study showed that Example Compositions C and D achieved an improved flavor satisfaction relative to the Comparative Compositions. Furthermore, Formulas B, C and D are found by the consumers to provide comparable whitening satisfaction relative to a commercial formula.

### Example 5

Fourier-transform infrared spectroscopy (FTIR) can be leveraged to examine changes in the environment around a molecule and interactions between molecules by measuring the vibrational frequencies. Polymers such as PVP contain moieties such as lactam amide that can readily participate in hydrogen bonding. Using FTIR, it is possible to determine whether hydrogen bonding is occurring by examining the vibrational shifts.

In this study, FTIR is applied to compare the 2%, 3%, and 4% hydrogen peroxide toothpaste formulas according to Example Compositions C, D and E, against the corresponding commercial cPVP-HP formula toothpaste with 2%, 3%, and 4% HP, respectively, as described in Example 4. Infrared spectra are collected using a Bruker Vertex 70 FTIR spectrometer (Bruker Optics, Billerica, MA) equipped with a GladiATR diamond ATR accessory (Pike Technologies, Madison, WI). The spectral range is 80-4000 cm⁻¹ and a resolution of 4 cm⁻¹ is used. All measurements are carried out at room temperature on as prepared toothpaste.

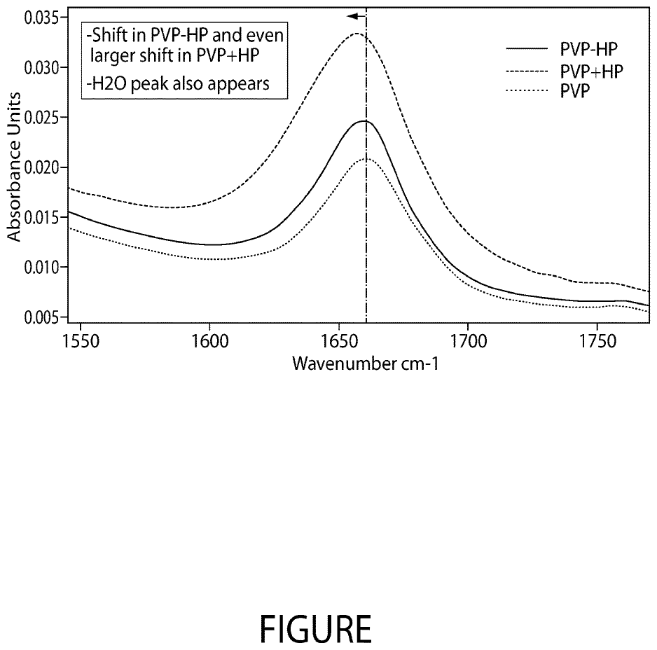

It is found that comparing each spectrum for toothpaste formulas having the same percentage of HP, the FTIR spectra are similar but not identical from 200 through about 4000 cm¹. Of most interest, is the C═O stretching vibration for the enol tautomer of the pyrrolidone amide group, which appears at about 1600 to 1700 cm⁻¹ (peaking at 1660 cm⁻¹). See FIG. 1. As shown by a comparison to PVP polymer, in both toothpaste formulas, the peak is shifted slightly to the left. In addition, in both toothpaste formulas, the peak reaches a significantly higher intensity compared to the same peak in PVP polymer. Surprisingly, the intensity of the peak in the compositions made according to the present disclosure, is substantially greater than that of the same peak in the corresponding compositions made from cPVP-HP complex. Thus, while similar, the FTIR spectra for the PVP pyrrolidone amide peak of the compositions according to the present disclosure are not identical to the same for the compositions formulated with cPVP-HP complex (see FIGURE). FIGURE is a FTIR spectra graph of Example Composition C, Comparative Composition 1, and a pure PVP polymer. Thus, these data suggest that there is in-situ formation of a PVP-HP complex in the compositions according to the disclosure, and that the precise nature of the complexing appears to be somewhat different from that seen in the compositions according to the prior art.

### Example 6

To test whether the order of addition of the ingredients is critical to the stability of the resulting formulations, two compositions having a formula substantially as shown for Formula C above (2% hydrogen peroxide) are prepared. In one formulation, the 50% aqueous hydrogen peroxide is added at the beginning of the process along with the propylene glycol humectant. In the other formulation, the 50% aqueous hydrogen peroxide is added at the end of the process, after all other ingredients were combined. The two compositions are then aged at a temperature 40° C. for 3 months, with active oxygen content measured at 1, 2, and 3 months. It is found that the same active oxygen levels are achieved in both compositions at each of the three time points:

| | 40° C., 1 month | | 40° C., 2 months | | 40° C., 3 months | |
|---|---|---|---|---|---|---|
| | HP added | HP added | HP added | HP added | HP added | HP added |
| | upfront | at the end | upfront | at the end | upfront | at the end |
| 2 wt. % HP | 2.0 wt. % | 2.0 wt. % | 2.0 wt. % | 2.0 wt. % | 1.9 wt. % | 1.9 wt. % |

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. An aqueous abrasive oral care composition comprising (a) 1-10 wt. % hydrogen peroxide, (b) 5-20 wt. % of calcium pyrophosphate abrasive, (c) 35-75 wt. % propylene glycol, (d) 5-20 wt. % of a thickening system, the thickening system comprising from about 6 to about 16 wt. % of a polyvinylpyrrolidone and from about 2 to about 4 wt. % of a thickening silica, (e) 1-20 wt. % of a polyethylene glycol/polypropylene glycol random copolymer;
provided that:
(1) the hydrogen peroxide is incorporated into the composition as a 40-80% aqueous solution; or
(2) the hydrogen peroxide is incorporated into the composition as a 30-80% aqueous solution and the composition is not formulated with metal ion chelating agents selected from metal stannate salts, colloidal stannate, EDTA, and salts thereof,
wherein the oral care composition has a weight ratio of the polyvinylpyrrolidone to the silica thickeners of about 5:1 to about 1:1 and the oral care composition has a viscosity of about 50,000 to 250,000 cP at room temperature using Brookfield viscometer and a spindle no. 3 for 1 minute.

2. The oral care composition according to claim 1, wherein the hydrogen peroxide is incorporated into the composition as an about 40-80% aqueous solution, e.g., an about 40-70% aqueous solution, or an about 40-60% aqueous solution, or an about 45-55% aqueous solution.

3. The oral care composition according to claim 1, wherein the oral care composition has a pH of about 5.5 to about 7.

4. The oral care composition according to claim 1, wherein the oral care composition has a viscosity of about 150,000 to about 250,000 cP, at room temperature using Brookfield viscometer and a spindle no. 3 for 1 minute.

5. The oral care composition according to claim 1, wherein the composition comprises about an equal amount of water and hydrogen peroxide by weight.

6. The oral care composition according to claim 1, wherein the composition is not formulated using a pre-formed PVP-hydrogen peroxide complex that is added to the composition during manufacture.

7. The oral care composition according to claim 1, wherein the propylene glycol is present in the amount of 45-70 wt. %.

8. The oral care composition according to claim 1, wherein the composition does not comprise glycerol.

9. The oral care composition according to claim 1, wherein the propylene glycol is the only polyol humectant in the oral care composition.

10. The oral care composition according to claim 1, wherein the polyvinylpyrrolidone is present in the amount of about 6 to about 14 wt. %.

11. The oral care composition according to claim 1, wherein the hydrogen peroxide and the polyvinylpyrrolidone form a polyvinylpyrrolidone-hydrogen peroxide complex in situ during manufacture of the composition.

12. The oral care composition according to claim 1, wherein the composition comprises 5 to 20 wt. % of a polyethylene glycol/polypropylene glycol (PEG/PPG) random copolymer.

13. The oral care composition according to claim 1, wherein the composition is not formulated with metal ion chelating agents.

14. The oral care composition according to claim 1, wherein the weight ratio of polyvinylpyrrolidone to hydrogen peroxide in the composition is 5:1 to 1.5:1.

15. The oral care composition according to claim 1, wherein the composition comprises 1-6 wt. % hydrogen peroxide, 1-6% water, 12-18 wt. % of calcium pyrophosphate abrasive, 40-60 wt. % propylene glycol, 8-14 wt. % polyvinylpyrrolidone, 5-18 wt. % of a random polyethylene glycol/polypropylene glycol random copolymer, 1-5 wt. % fumed silica, 1-5 wt. % of an anionic surfactant (e.g., sodium lauryl sulfate), 0.01-0.5% of an antioxidant (e.g., BHT); wherein the hydrogen peroxide is incorporated into the composition as an about 50% aqueous hydrogen peroxide solution.

16. An aqueous abrasive oral care composition comprising:

| Ingredient | Wt. % | |
|---|---|---|
| Propylene Glycol | 40-60% | (e.g., 45-55%) |
| Aqueous Hydrogen Peroxide | 1-10% | (e.g., 1-6%) |
| Solution (50 wt. % Hydrogen | | |
| peroxide) | | |
| cPVP | 6-18% | (e.g., 8-14%) |
| Sodium Lauryl Sulfate | 1-3% | (e.g., 2%) |
| Fumed Silica | % | (e.g., 1.5-5.5%) |
| Calcium Pyrophosphate | 5-20% | (e.g., 8-18%) |
| Tetrasodium Pyrophosphate | 1-5% | (e.g., 1-2%) |
| (TSPP) | | |
| Disodium Pyrophosphate (DSPP) | 0.1-2% | (e.g., 0.25-1%) |
| Sodium Monofluorophosphate | 0.5-2% | (e.g., 0.75-1.2%) |
| PEG/PPG-116/66 Copolymer | 5-15% | (e.g., 7.5-10%) |
| Flavoring Agents | 0.1-4% | |
| Preservative(s) | 0-0.05% | (e.g., 0.02-0.03%) |

17. The oral care composition according to claim 1, wherein the composition does not comprise polyethylene glycol, poloxamer, or polycarboxymethylene polymer.

18. A method of whitening a tooth surface, cleaning an oral cavity surface, and/or treating, preventing or ameliorating a disease, disorder or condition of the oral cavity, the method comprising the step of applying an effective amount of the oral care composition according to claim 1, to an oral cavity surface of a subject in need thereof.

19-20. (canceled)
